(19) United States
(12) Patent Application Publication
(10) Pub. No.: US 2004/0115092 A1
(43) Pub. Date: Jun. 17, 2004

(54) Caffeine detector

(76) Inventor: Paul Starr, Englewood Cliffs, NJ (US)

Correspondence Address: Michael G. Petit, P. O. Box 91929, Santa Barbara, CA, 93190-1929, US

(21) Appl. No.: 10/322,090

(22) Filed: Dec. 17, 2002

(51) Int. Cl. G01N 33/53; G01N 21/77; G01N 21/78

(52) U.S. Cl. 422/55; 422/57; 422/61; 544/274; 436/164; 435/7.1; 435/975

(57) ABSTRACT

A kit for determining the presence of caffeine in a beverage. The kit is portable, provides a rapid, visible indication of the presence of caffeine in the beverage and may be read without the use of instrumentation. A first embodiment of the kit comprises (a) a probe adapted to be dipped into the beverage, a portion of the probe having an antibody affixed thereto operable for specifically binding to either caffeine or a labeled protein-caffeine conjugate; (b) a wash solution comprising water; (c) a solution comprising a labeled protein-caffeine conjugate. The label comprising the protein-caffeine conjugate is preferably a chromatophore that is visible to the eye. In another embodiment, the kit further includes a developer reagent. The label is made visible by reacting the probe bearing the protein-caffeine conjugate with a developer reagent to provide a visible product.

### BACKGROUND OF THE INVENTION

1. Field of the Invention

The present invention relates to a test kit for detecting the presence of caffeine in a fluid beverage.

2. Prior Art

Beverages that contain caffeine may have adverse health effects on certain individuals. For example, pregnant women and persons having ulcers or other gastrointestinal disorders are routinely advised to avoid caffeine in foods and beverages. Moreover, it is well known that caffeine may act as a stimulant and may interfere with the normal sleeping process. Accordingly, many individuals deliberately order decaffeinated beverages from restaurants and the like, especially in the evening or at night. However, in busy eating or drinking establishments, a caffeinated beverage may occasionally mistakenly be served despite the placing of an order for a decaffeinated beverage. The presence of caffeine in a beverage is usually undetectable by an individual at the time of consumption. If the individual is mistakenly served a caffeinated beverage, the person may experience the delayed effect of sleeplessness or anxiety. Such inadvertent ingestion of caffeine is also undesirable for pregnant women, persons having gastrointestinal disorders, and others who have been medically advised to avoid the ingestion of caffeine.

Various methods of determining the presence of methylxanthine compounds such as caffeine are known to those skilled in the art. For example, a method of measuring the concentration of theophylline, a compound chemically related to caffeine, in whole blood using an immunochromatographic stick is described in Elias-Jones, A. C. et al., Arch. Dis. Child. 62/8 (1987), pp. 836-54. Thin layer chromatography (TLC) has also been employed to detect caffeine in samples. However, such techniques are either inconvenient or difficult to employ in other than a laboratory environment.

In view of the foregoing, it would be useful to be able to employ a simple method and device to detect the presence of caffeine. It would be particularly useful to be able to provide a portable device for such detection which does not require instrumental means to interpret the result, as this would enable the user to readily determine whether beverages served at restaurants and the like are decaffeinated.

### SUMMARY

It is a primary object of the invention to provide a simple device and method for the detection of caffeine in a fluid. It is a significant feature of the device and the method of the present invention that the device is portable and that the method for using the device to determine the presence (or absence) of caffeine in a beverage may be used in restaurants without attracting attention to the tester.

The present invention discloses a kit to be used by a person to detect the presence or absence of caffeine in a beverage. The kit includes, as essential elements, a solid support adapted to contact, either directly or indirectly, the beverage, and a chromogenic visualization reagent. The probe provides a solid support for caffeine-specific antigens and/or caffeine-specific antibodies conjugated to discrete portions thereof. The kit is used by a person to detect the presence of caffeine in a beverage by: (a) contacting the probe with the beverage; then (b) washing the probe, and (c) applying a chromogenic visualizing reagent to the probe to identify the presence of caffeine thereon. The kit is useful for the self-detection and identification of any one of a plurality of possible happens in a beverage or food.

The kit includes a probe having one or more discrete portions with antigen-specific antibodies bound thereto; such antibodies binding caffeine-bound antigens which are particular to and characteristic of a hapten such as caffeine. If a particular hapten is present within the beverage, the hapten will adhere to a corresponding portion of the probe. The probe is then exposed to an appropriate reagent causing a visually perceivable signal. The visual signal is present only on a discrete portion of the probe where an antigen-specific antigen-antibody complex is present. Such a visual signal may then be interpreted by a person.

It is, therefore, the primary object of the present invention to provide a diagnostic kit which may be used by a person for identifying the presence of caffeine in a beverage. The features of the invention believed to be novel are set forth with particularity in the appended claims. However the invention itself, both as to organization and method of operation, together with further objects and advantages thereof may be best understood by reference to the following description taken in conjunction with the accompanying drawings in which:

### BRIEF DESCRIPTION OF THE DRAWINGS

FIG. 1(A) shows a probe having a discrete indicator portion thereon to which specific antibodies are conjugated that are operable for binding to caffeine or a protein-caffeine conjugate being dipped into a beverage.

FIG. 1(B) shows the probe of FIG. 1 with caffeine conjugated thereto being dipped into a wash solution to remove unbound caffeine.

FIG. 1(C) shows the probe of FIG. 1(B) being dipped into a solution comprising a labeled protein-caffeine conjugate.

FIG. 1(D) shows the probe being dipped into a wash solution to remove any unbound labeled protein-caffeine conjugate therefrom.

FIG. 2(A) shows the probe wherein the absence of the visually perceivable labeled protein-caffeine conjugate on the indicator portion of the probe indicates the presence of caffeine in the beverage.

FIG. 2(B) shows the probe wherein the presence of the visually perceivable labeled protein-caffeine conjugate on the indicator portion of the probe indicates the absence of caffeine in the beverage.

### DESCRIPTION OF THE PREFERRED EMBODIMENTS

A hapten is a substance that can react with an antibody but cannot cause an immunological response. Caffeine, a hapten, can be covalently linked to a protein, and the resulting protein-caffeine conjugate used to immunize animals. The specific antibody recovered from the serum of the immunized animal will bind specifically to either caffeine or the protein-caffeine conjugate. In addition, the specific antibody may then be bound to a particular region of a support or probe such as, for example, a paper strip, by methods for immobilizing antibodies on a support that are well known in the art. If the probe is dipped into a beverage comprising caffeine, the caffeine will bind to the specific antibody on the probe. If the binding site(s) of the antibody are occupied by caffeine, and a second “developer” antigen comprising a labeled protein-caffeine conjugate is contacted with the probe, the labeled conjugate will not adhere to the indicator portion of the probe to which specific antibody is bound and can be washed off. Thus, the absence of the label on the indicator portion of the probe indicates the attachment of caffeine thereto and, accordingly, the presence of caffeine in the beverage.

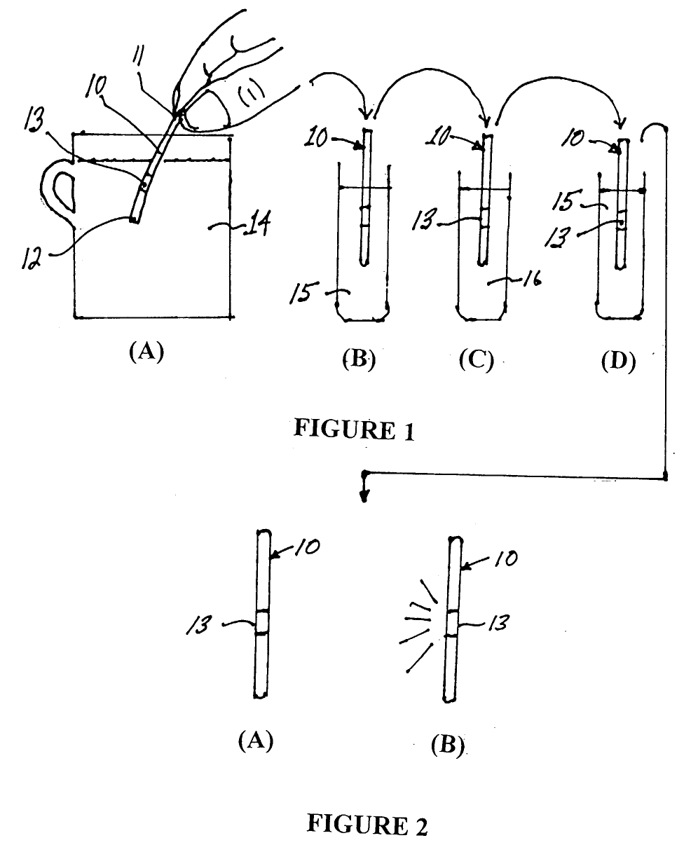

Turning first to FIG. 1A, a probe 10 has a proximal end 11 and a distal end 12. The distal end 12 has an indicator portion 13 adjacent thereto, which indicator portion is coated with specific antibody. The probe 10 is brought into contact with a beverage 14, either by directly inserting the probe into the beverage, or by inserting the probe into an appropriately buffered diluent solution containing a sample of the beverage. FIG. 1(B) illustrates the probe after contacting the probe with the beverage. If there is caffeine present in the beverage, the indicator portion 13 of the probe, which is coated with antibody, will comprise caffeine by forming an antigen-antibody complex. FIG. 1(B) shows the probe 10 being washed in water 15 to remove unbound caffeine.

After the probe is washed to remove unbound caffeine, the probe 10 is dipped into a solution 16 containing labeled protein-caffeine conjugate. If there is caffeine in the beverage, the labeled protein-caffeine conjugate will not be able to bind to the antibody on the indicator region due to the lack of an available binding site. If there is no caffeine in the beverage, the labeled protein-caffeine conjugate will bind to the indicator portion. Accordingly, the presence of a visual signal on the indicator portion of the probe indicates that the beverage is caffeine-free.

A number of variations of the test kit are possible. The probe, which serves as a solid support for the caffeine-specific indicator portion, may be fabricated from a natural or synthetic polymer, a glass or a ceramic material. The anti-caffeine antibody bound to the probe may be monoclonal or polyclonal, monovalent or polyvalent. Caffeine in a beverage can be detected by contacting the probe with the beverage, washing unbound material from the probe, contacting the probe with a reagent comprising labeled carrier protein-caffeine conjugate, washing the probe a second time and visualizing the portion(s) of the probe having a antigen-antibody complex thereon. In the event that the label on the conjugate does not provide a visible indication of its presence on the indicator portion, the presence of the label on the indicator portion of the probe can be visualized by immersing the probe in a chromogenic developing reagent and direct observation. Appropriate color development (i.e. chromogenic) reagents and their uses for detecting the presence of an antigen-antibody complex are presented, for example, in U.S. Pat. No. 5,447,837. Reporter enzymes such as peroxidase, and a peroxidase substrate, disclosed in U.S. Pat. Nos. 5,589,344 and 5,460,946, can also be employed as a chromogenic reagent. Such enzyme-substrate systems, however, require the use of buffers for pH regulation.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What I claim is:

1. A kit operable for providing a visual indication of the presence of caffeine in a beverage.

2. The kit of claim 1 comprising: (a) a probe adapted to be dipped into said beverage, a portion of said probe having an antibody affixed thereto operable for specifically binding to caffeine or a protein-caffeine conjugate; (b) a wash solution comprising water; (c) a solution comprising a labeled protein-caffeine conjugate wherein said label comprising said protein-caffeine conjugate is visible to the eye.

3. The kit of claim 1 comprising: (a) a probe adapted to be dipped into said beverage, a indicator portion of said probe having an antibody affixed thereto operable for specifically binding to caffeine or a protein-caffeine conjugate; (b) a wash solution comprising water; (c) a solution comprising a labeled protein-caffeine conjugate wherein said label comprising said protein-caffeine conjugate is not visible; and (d) a solution comprising a developer reagent that produces a visible product in the presence of said labeled protein-caffeine conjugate.

4. A method for detecting the presence of caffeine in a beverage comprising the steps of:
(a) presenting a beverage; then
(b) presenting a kit in accordance with claim 2; then
(c) dipping said probe into said beverage such that said indicator portion of said probe contacts said beverage; then
(d) removing said probe from said beverage and dipping said probe into said water in order to remove unreacted caffeine from said probe; then
(e) dipping said probe into said solution comprising said labeled protein-caffeine conjugate; then
(f) washing unbound labeled protein-caffeine conjugate from said probe by dipping said probe into said water; then
(g) visually inspecting said probe, the presence of a visible label on said indicator portion of said probe indicating the absence of caffeine in said beverage.
